(19) United States
(12) Patent Application Publication
(10) Pub. No.: US 2024/0099821 A1
(43) Pub. Date: Mar. 28, 2024

(54) TIBIA-CALCANEUS TRUSS

(71) Applicant: Kyon AG, Zürich (CH)

(72) Inventor: Slobodan TEPIC, Zürich (CH)

(73) Assignee: Kyon AG, Zürich (CH)

(21) Appl. No.: 17/768,586

(22) PCT Filed: Oct. 14, 2020
(86) PCT No.: PCT/EP2020/078889

§ 371 Date: Apr. 13, 2022

(51) Int. Cl. A61D 1/00; A61F 2/42

(52) U.S. Cl. CPC A61D 1/00; A61F 2/4202; A61F 2002/307; A61F 2002/4217

(57) ABSTRACT

The present invention provides an implant in a form of a truss, spanning the distance from diaphysis of the tibia to the tip of the calcaneus, thus fixing the ankle (hock) joint in extension. Gastrocnemius muscles are thus at close to their shortest length and their force capacity is greatly reduced, preventing the caudal luxation of the distal femur even if the cranial cruciate ligament is torn.

### FIELD OF THE INVENTION

The present invention relates to a surgical implant useful in treating disorders of the knee, termed the stifle in dogs, which fixes the hock joint of the dog in extension and thus reduces the pull of the gastrocnemius muscles on the femur, which is the main destabilizing force causing caudal dislocation of the femoral condyles if the cranial cruciate ligament is ruptured.

The implant can also be used to treat other pathological conditions of the distal hind limb in dogs and cats, e.g. of arthrosis of the hock joint or of the rupture of the Achilles tendon.

### Background of the Invention

The anterior cruciate ligament (ACL) in the human knee joint, commonly called the cranial cruciate ligament (CrCL) in the canine stifle, is frequently torn in trauma. It also frequently fails, particularly in dogs, after a degenerative process of still unknown etiology.

In human orthopedics, standard procedures replace the failed ACL with an ACL cadaveric allograft or with an autograft made from a part of the patient's own patellar tendon or a part of the fascia and tendon removed from the hamstring muscles. The procedure results in a stable knee, but the long-term performance of the knee is often unsatisfactory. Roughly 75-90% of cases result in degenerative arthritis of the joint within 15 years of the procedure.

In dogs, the standard procedures involve either placement of an extra-capsular suture or performance of one of several geometry-modifying surgical techniques. In the extra-capsular procedure, a suture is placed outside of the joint, usually on the lateral side, to approximate the function of the CrCL. The intention of the suture application is to provide stability of the joint for several weeks while waiting for fibrosis to occur around the joint. This fibrosis should then provide for long-term stability. However, the extra-capsular suture technique regularly results in failure. Degenerative arthritis of the joint, after a year or so, is the rule rather than the exception.

Attempts to replace the CrCL in the dog by an anatomically placed, intra-articular artificial ligament have also generally failed in spite of years of research and development of materials, anchor designs, and surgical techniques.

In surgical, geometry-modifying techniques, the tibia is cut and a segment of it is repositioned to change the geometry of the tibia and/or the joint in order to stabilize the stifle. Various techniques have been used, including: tibial plateau leveling osteotomy (TPLO; see U.S. Pat. No. 4,677,973 and Slocum and Slocum, Vet. Clin. North Am. 23:777-795, 1993), cranial closing wedge osteotomy (CWO; Slocum and Devine, J. Am. Vet. Med. Assoc. 184:564-569, 1984), and tibial tuberosity advancement (TTA; Tepic et al., Biomechanics Of The Stifle Joint, in Proceedings of the 1st World Orthopaedic Veterinary Congress, Munich, Germany, pp. 189-190. 2002). Of the surgical approaches used in dogs, TTA seems to be associated with less morbidity and faster recovery, and it also provides immediate and durable stability to the joint (Boudrieau, Vet Surg., 38(1):1-22, 2009). Nevertheless, surgical complications are not uncommon with all these techniques. The most common is post-surgical damage to the medial meniscus caused by excessive, supra-physiological movement between the femur and the tibia.

### SUMMARY OF THE INVENTION

The present invention provides an implant in a form of a truss, spanning the distance from diaphysis of the tibia to the tip of the calcaneus, thus fixing the ankle (hock) joint in extension. Gastrocnemius muscles are thus at close to their shortest length and their force capacity is greatly reduced, preventing the caudal luxation of the distal femur even if the cranial cruciate ligament is torn.

The present invention provides an implant and methods for surgically treating a disordered knee in a dog by fixing the hock (ankle) joint in extension. The disorder can be a partial or complete rupture of the cranial cruciate ligament (CrCL) due to any circumstance (e.g., due to trauma or a disease process), and the methods of the invention can also be applied to treat other orthopedic problems such as arthrosis of the hock joint or trauma-related problems, e.g. rupture of the Achilles tendon. The conventional methods now used for arthrodesis of the hock joint are technically demanding and have high complication rates. The same is true for repairs of the Achilles tendon—mandatory temporary external restraints by either splints or even external fixation across the joint are prone to many serious complications.

The implant and methods of this invention emerged from our study of the probable causes of stifle joint instability following the rupture of the CrCL. The main flexors of the stifle are hamstrings bridging both the hip joint and the knee joint from their origins at the caudal pelvis to their insertions at the proximal tibia and the gastrocnemius muscles, bridging the knee and the hock, from their origins at the distal femur and insertion at the calcaneus of the tarsus. In addition to being flexors of the knee, the hamstrings are extensors of the hip joint; the gastrocnemius muscles are flexors of the knee and extensors of the hock. The main antagonists to these two groups of knee flexors are quadriceps muscles inserting on the patella and crossing over to the tibia. Three of the quad muscles originate at the proximal femur and one on the pelvis.

Intricate balance of these muscles and their roles in the stifle mechanics have not been scrutinized in sufficient detail until our recent ex vivo study on dog hind limbs that has provided clear and strong evidence that reducing or eliminating pull of the gastrocnemius muscles onto the distal femur can yield a stable knee even if the cranial cruciate is transected.

Hamstrings are stabilizing the CrCL deficient knee, but if the hock joint is fixed in extension and the need for the pull of the gastrocnemius muscles thus eliminated (to prevent flexion of the hock under load), the pull of the quad muscles, needed to maintain the knee extended, will maintain the knee joint in its normal, reduced position.

An implant in a form and with a function of a truss that fixes the distal tibia to the caudal aspect of the calcaneus can maintain nearly normal functionality of the distal limb without pain and lameness caused by cranial cruciate ligament rupture and its sequelae. Conventional surgeries for the fusion of the hock joint could accomplish the same result, but those surgeries require high skill and involve extensive dissection of the tissues and use of implants prone to failures. The current invention allows for a minimally invasive approach with lesser surgical skill required, lower risks and a faster post-surgical recovery.

Accordingly, the present invention features a surgical implant and methods of its use in treating a disordered dog stifle by surgically immobilizing the hock joint in extension, hence greatly diminishing the maximum force that gastrocnemius muscles can exert on the distal femur. As noted, the stifle pathology may include a partially or fully ruptured cranial cruciate ligament, which we may refer to as cranial cruciate ligament disease. Disorders of the dog distal hind limb also amenable to treatment with this same implant are arthrosis of the hock and rupture of the Achilles tendon.

### BRIEF DESCRIPTION OF THE DRAWINGS

FIG. 1 is a sagittal view of the dog hind limb bones—the muscles are shown only schematically.

FIG. 2 is a sagittal view of the dog hind limb bones with the implant of the invention affixed as a truss from the distal tibia to the caudal aspect of the calcaneus.

FIG. 3 shows the truss in orthogonal views.

FIG. 4 shows the truss and the locking screws in perspective views from mainly medial and lateral directions.

FIGS. 5(a) and (b) show the truss with three and two locking screws, respectively for fixation to the calcaneus.

### DETAILED DESCRIPTION

This invention is based, at least in part, on ex vivo experiments and clinical observations that have helped us identify the fundamental causes of the stifle instability. Because the basic geometry of the bones articulating at the stifle is that of four convex, incongruent surfaces—the medial and lateral condyles of the femur in contact with the medial and lateral condyles of the tibia—the joint is fundamentally unstable and prone to subluxations. It is kept together and in alignment with collateral ligaments on the medial and lateral sides of the condyles and the cruciate ligaments inside the joint. The cranial cruciate rupture or even its partial rupture allows for excessive movements between the articulating surfaces of the femur and the tibia and thus leads to damage and ultimately destruction of the menisci, which act as gaskets of the joint limiting the rate of exudation of the fluid from opposing, poorly congruent layers of cartilage. Instability of the joint subluxating with every step causes mechanical lameness and ultimately leads to arthrosis of the joint and persistent pain.

The main muscle groups bridging the stifle are: (i) extensors (quadriceps: vastus lateralis, vastus medialis and vastus intermedius, with rectus femoris), (ii) flexors attached to the proximal tibia (hamstrings: biceps femoris, semitendinosus, semimembranosus plus gracilis and sartorius) and (iii) flexors attached to the femur (medial and lateral gastrocnemius). In standing and in gait quadriceps must act to prevent flexion at the stifle, gastrocnemius must act to prevent flexion of the hock and hamstrings must act to prevent flexion of the hip (at the least to balance the pull of the rectus femoris). Due to their attachments and direction of action, muscles of the group (ii), mainly hamstrings, act to prevent caudal subluxation of the femoral condyles over the tibial condyles, while muscles of the group (iii) are the main drivers of the subluxation. A solution to the conundrum which is the basis of this invention is in limiting the force of the muscles of the group (iii) by fixing the hock joint in extension, thus bringing these muscles to their shortest length and lowest force-generating capacity (Rassier D. E., Macintosh B. R., and Herzog W., Length dependence of active force production in skeletal muscle, J Appl Physiol (1985). 1999 May; 86(5):1445-57).

Our experimental work with dog cadavers has shown a surprisingly strong stabilizing effect of eliminating the pull of the muscles of the group (iii) onto the femur (via femoral fabellae), while adding the pull of the muscles of the group (ii) to the necessary pull of the muscles of the group (i) to prevent flexion of the stifle under simulated external loading. Strong equilibrium position of the stifle with transected cranial cruciate ligament and the intact caudal cruciate ligament under tension was established when the hock joint was fixed in extension by a truss from the distal tibia to the caudal aspect of the calcaneus. Even without the pull of the muscles of the group (ii) the joint was stable, but the stability was increased by increasing the relative force of these muscles in comparison to the loading force (which automatically defines the force of quadriceps).

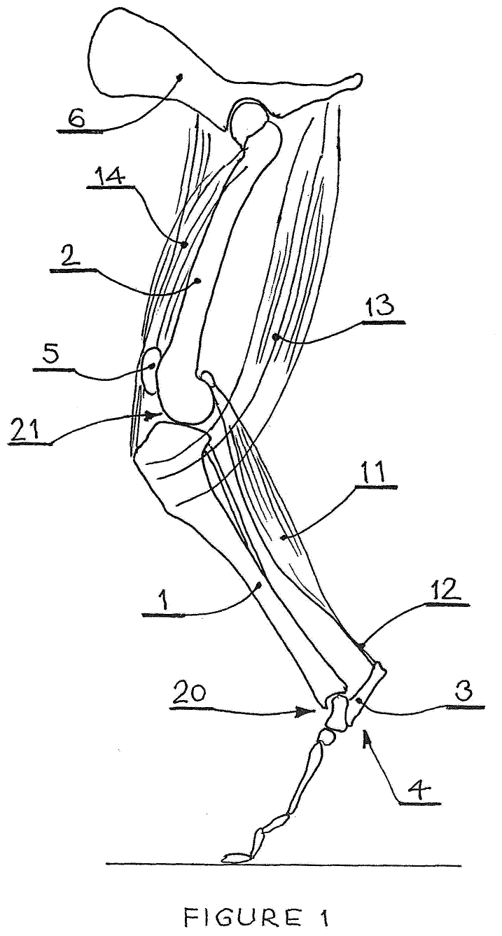

FIG. 1 shows the bones of the dog hind limb and the muscle groups responsible for preventing the collapse of the leg in standing position. The tibia 1 connects the hock joint 20, where the tibia articulates with the tarsus 4, to the stifle joint 21. The femur 2 connects the stifle joint 21 to the hip joint 22, where the femur articulates with the pelvis 6. Gastrocnemius muscles 11 originate at the distal femur and insert at calcaneus 3 via so-called Achilles tendon 12. They prevent flexion at the hock joint in standing position. Quadriceps 14 via patella 5, which transfers forces of the quadriceps to the tibia via patellar tendon, prevent flexion at the stifle joint 21. Hamstrings 13 hold the hip joint 22 in extension. Without tensile forces in these three muscle groups, the leg would collapse to the ground.

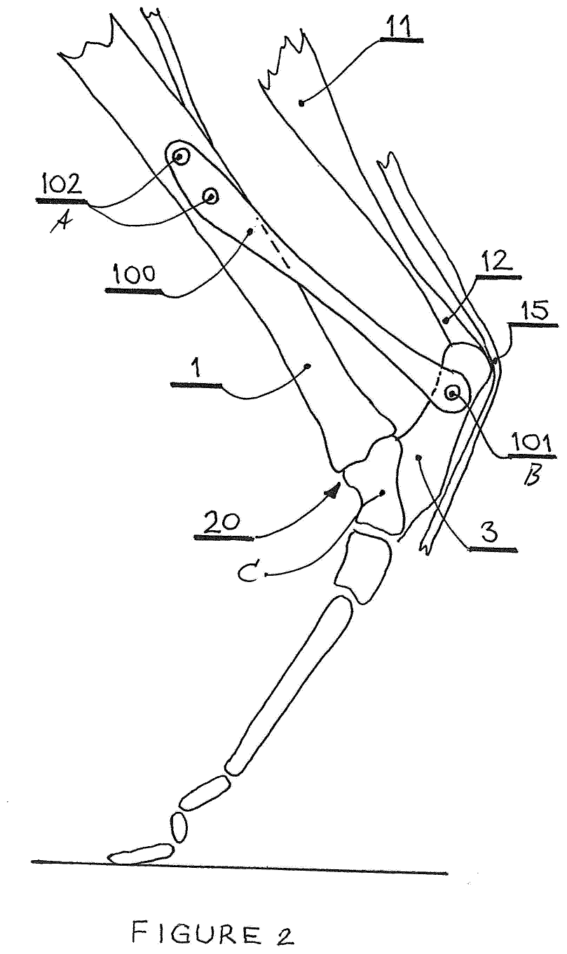

FIG. 2 shows the implant of this invention 100, as a truss spanning the hock joint 20 by fixation to the tibia 1 by screws 102 and to the calcaneus 3 by a screw 101. With the hock 20 in extension, as shown, gastrocnemius muscles 11, inserting to the calcaneus via tendons 12 cannot exert full force. The superficial digital flexor tendon 15 wraps around the calcaneal process or the tip of the calcaneus 3. Here, the truss is adapted to span the hock joint distant therefrom such that the proximal and distal ends of the truss and hock joint represent apexes A, B, C of a substantially triangular configuration.

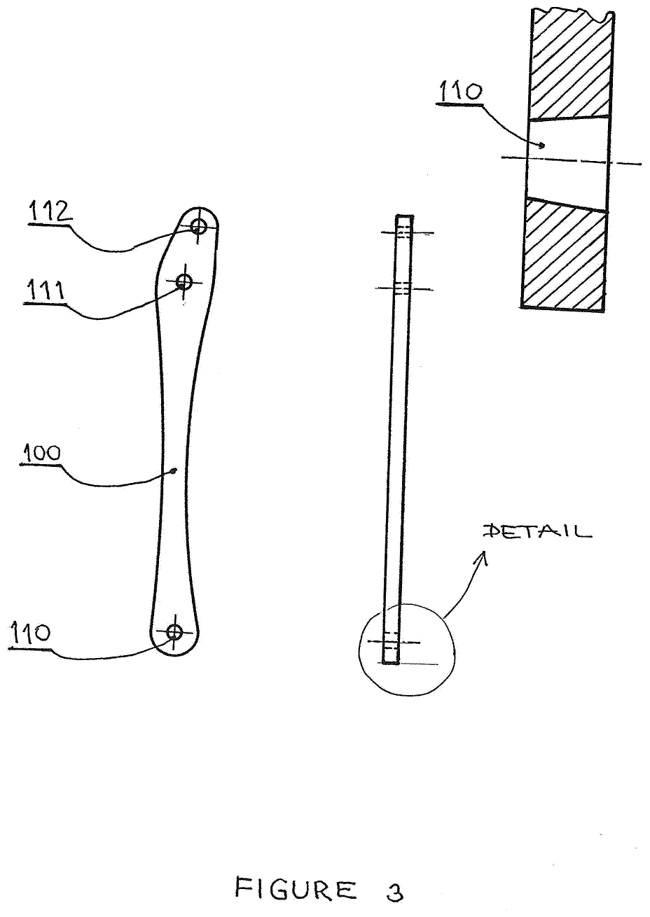

FIG. 3 shows orthogonal views of the truss 100. In one of the embodiments, there are two screw holes 111 and 112 proximally for fixation to the tibia and one screw hole 110. Detail of the distal end of the truss 100 in a cross-section, shows a conical hole 110 that accommodates the conical head of the locking bone screw 101.

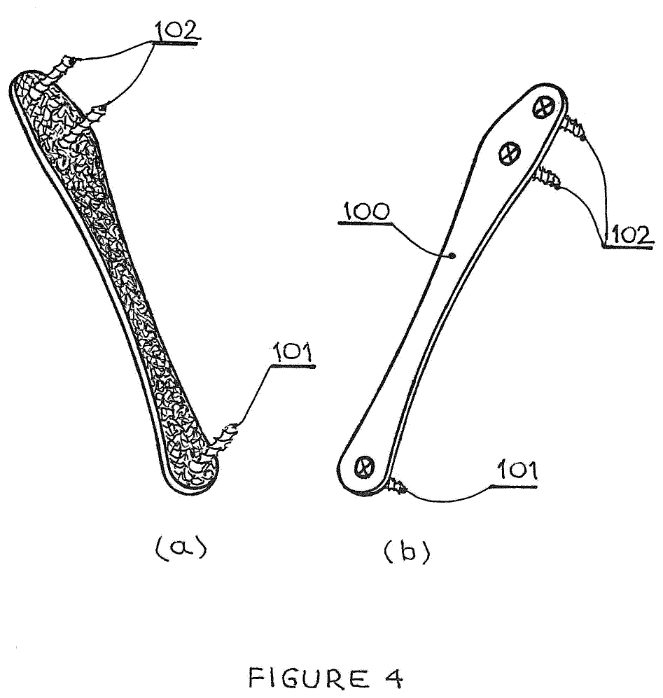

FIG. 4 shows perspective views of the truss 100 with screws 101 and 102 protruding from the bone-facing side of the truss 100. Since the function of the implant 100 is to be permanent, the bone-facing surface of the truss 100 may be coated for bone ingrowth, with for example porous titanium and/or hydroxyapatite. This can be done on the whole implant length or just at the end sections that make direct contact to bones.

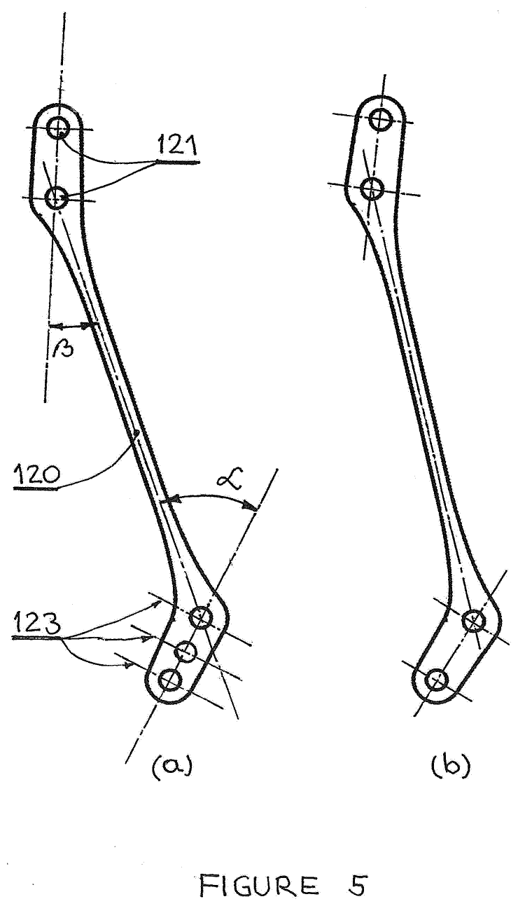

FIG. 5(a) shows another embodiment 120 of the truss with three holes 123 on the distal end for fixation to the calcaneus. Preferably, at least one of the proximal and distal ends of the truss is angled (see angles alpha and beta) with respect to the extension direction of the truss in adaptation to the respective extension direction of the tibia and of the calcaneus.

FIG. 5(b) shows a presently preferred modification of FIG. 5(a) with two holes on the distal end.

Clinical experience on a large number of dogs may provide firmer guidelines on the number of screws. At this time, the combination with two mono-cortical locking screws proximally and one bi-cortical screw passing through the full thickness of the very strong bone of calcaneus is considered an optimal solution.

The truss 100 and the bone screws 101 and 102 are preferably made of titanium or titanium alloys, such as Ti6Al4V or Ti6Al7Nb. Alternative materials are conventional implant steels, e.g. 316L. The truss and/or the screws may be coated for improved bony integration. Porous titanium and/or hydroxyapatite are well suited for that purpose.

Other than the use of this implant and the surgical procedure for cruciate disease, it is also well suited as an augmentation device for repairs of the tendon of Achilles as well as for hock joint arthrodesis.

Having disclosed at least one embodiment of the present invention, variations will be understood by one of ordinary skill in the art. Such adaptations, modifications, and improvements are considered part of the invention.

The following embodiments of the specification shall further characterize the invention without limiting its scope.
1 An implant truss for spanning the hock joint of a dog, by affixing the truss on its proximal end to the tibia and on its distal end to the calcaneus.
2. An implant truss according to embodiment 1, comprising at least one bone locking screw to be used on each end of the truss to affix the truss to the tibia and to the calcaneus.
3. An implant truss according to embodiment 1, comprising two mono-cortical locking screws with conical self-locking tapered heads to be used proximally in the tibia and one bi-cortical screw with the self-locking tapered head to be used distally in the calcaneus.
4. An implant truss according to embodiment 1, comprising two mono-cortical locking screws with conical self-locking tapered heads to be used proximally in the tibia and up to three bi-cortical screws with the self-locking tapered head to be used distally in the calcaneus.
5. An implant truss according to embodiment 1 made of titanium or titanium alloys.
6. An implant truss according to embodiment 1 coated for bony integration with porous titanium.
7 An implant truss according to embodiment 1 coated with hydroxyapatite.
8. An implant truss according to embodiment 1, wherein the truss is substantially rod shaped.
9. An implant truss according to embodiment 1, wherein at least one of the proximal and distal ends of the truss is angled with respect to the extension direction of the truss in adaptation to the respective extension direction of the tibia and of the calcaneus.
10. An implant truss according to embodiment 1, wherein the truss is adapted to span the hock joint distant therefrom such that the proximal and distal ends of the truss and the hock joint represent apexes of a substantially triangular configuration.
11. An implant truss according to embodiment 1, comprising two locking screws to be used proximally in the tibia and one screw to be used distally in the calcaneus.
12. An implant truss according to embodiment 1, comprising two locking screws to be used proximally in the tibia and up to three screws to be used distally in the calcaneus.
13. An implant kit for immobilizing the hock joint in extension comprising a truss and screws for affixing the truss to the tibia and to the calcaneus.
14. An implant kit according to claim 13, including the truss of embodiment 1.
15. An implant kit according to embodiment 13 sterile packaged.
16. A surgical intervention for cruciate deficient stifle comprising immobilization of the hock joint in extension.
17. A surgical intervention according to embodiment 16, using the implant truss of embodiment 1.
18. A surgical intervention according to embodiment 16 using the implant kit of embodiment 13.

1. An implant truss for spanning the hock joint of a dog, comprising a proximal end adapted for affixing to the tibia and a distal end adapted for fixing to the calcaneus.

2. The implant truss according to claim 1, comprising at least one hole on each end for inserting a bone locking screw to affix the truss to the tibia and to the calcaneus.

3. The implant truss according to claim 1, comprising two holes for inserting mono-cortical locking screws with conical self-locking tapered heads proximally in the tibia and one hole for inserting a bi-cortical screw with a self-locking tapered head distally in the calcaneus.

4. The implant truss according to claim 1, comprising two holes for inserting mono-cortical locking screws with conical self-locking tapered heads proximally in the tibia and up to three holes, e.g. 1, 2 or 3 holes for inserting bi-cortical screws with a self-locking tapered head distally in the calcaneus.

5. The implant truss according to claim 1, made of titanium or titanium alloys.

6. The implant truss according to claim 1, coated with a material for bony integration.

7. The implant truss according to claim 1, coated with porous titanium and/or hydroxyapatite.

8. The implant truss according to claim 1, wherein the truss is substantially rod shaped.

9. The implant truss according to claim 1, wherein at least one of the proximal and distal ends of the truss is angled with respect to the extension direction of the truss in adaptation to the respective extension direction of the tibia and of the calcaneus.

10. The implant truss according to claim 1, wherein the truss is adapted to span the hock joint distant therefrom such that the proximal and distal ends of the truss and the hock joint represent apexes of a substantially triangular configuration.

11. The implant truss according to claim 1, comprising holes for inserting two locking screws proximally in the tibia and one hole for inserting a screw distally in the calcaneus.

12. The implant truss according to claim 1, comprising holes for inserting two locking screws proximally in the tibia and holes for inserting up to three screws, e.g. 1, 2 or 3 screws, distally in the calcaneus.

13. An implant kit for immobilizing the hock joint, particularly the hock joint of a dog in extension comprising a truss and screws for affixing the truss to the tibia and to the calcaneus.

14. The implant kit according to claim 13, comprising an implant truss for spanning the hock joint of a dog, comprising a proximal end adapted for affixing to the tibia and a distal end adapted for fixing to the calcaneus.

15. The implant kit according to claim 13, which is sterile packaged.

16. A surgical intervention method for cruciate deficient stifle comprising immobilizing the hock joint in extension.

17. The surgical intervention method according to claim 16, comprising implanting a truss for spanning the hock joint of a dog, wherein said truss comprises a proximal end adapted for affixing to the tibia and a distal end adapted for fixing to the calcaneus.

18. The surgical intervention method according to claim 16, comprising immobilizing the hock joint of a dog in extension using an implant kit, wherein said implant kit comprises a truss and screws for affixing the truss to the tibia and to the calcaneus.
